(19) United States
(12) Patent Application Publication
(10) Pub. No.: US 2025/0057647 A1
(43) Pub. Date: Feb. 20, 2025

(54) PRODUCTION OF A VALVE IMPLANT AND USE THEREOF

(71) Applicants: Université de Bordeaux, BORDEAUX (FR); Institut National de la Santé et de la Recherche Médicale, PARIS (FR); Centre Hospitalier Universitaire de Bordeaux, TALENCE (FR); Fondation Bordeaux Université, BORDEAUX (FR)

(72) Inventors: Fabien KAWECKI, SAINT-MEDARD-EN-JALLES (FR); Nicolas L’HEUREUX, TALENCE (FR); Jean-Benoît THAMBO, BORDEAUX (FR); François ROUBERTIE, VILLENAVE D'ORNON (FR)

(21) Appl. No.: 18/723,133

(22) PCT Filed: Dec. 21, 2022
(86) PCT No.: PCT/FR2022/052464

§ 371 Date: Jun. 21, 2024

(30) Foreign Application Priority Data
Dec. 23, 2021 (FR) FR2114305

(51) Int. Cl. A61F 2/24; A61L 27/36; A61L 27/50

(52) U.S. Cl. CPC A61F 2/2415; A61L 27/3633; A61L 27/507; A61F 2220/0075; A61L 2430/20

(57) ABSTRACT

The present invention relates to the production of a valve leaflet implant and to the use thereof in the treatment of congenital heart disease, cardiac, venous and lymphatic valvulopathies, in particular the tetralogy of Fallot.

### CROSS-REFERENCE TO RELATED APPLICATIONS

The present application is a filing under 35 U.S.C. 371 as the National Stage of International Application No. PCT/FR2022/052464, filed Dec. 21, 2022, entitled “PRODUCTION OF A VALVE IMPLANT AND USE THEREOF,” which claims priority to France Application No. 2114305 filed with the Intellectual Property Office of France on Dec. 23, 2021, both of which are incorporated herein by reference in their entirety for all purposes.

### TECHNICAL FIELD OF THE INVENTION

The present invention relates to the production of a valve leaflet implant and to the use thereof in the treatment of congenital heart disease, cardiac, venous and lymphatic valvulopathies, in particular the tetralogy of Fallot.

In the following description, the references between brackets [symbol] refer to the list of references presented at the end of the text.

### STATE OF THE ART

A congenital heart disease is a malformation of the heart present at birth. Its severity can vary, from a very minor form that will never cause heart problems, to a very serious form requiring treatment. Congenital heart disease occurs when the chambers, walls or valve leaflets of the heart—or the blood vessels near the heart—do not develop normally before birth. The majority of congenital heart diseases can be classified into two broad categories: cyanogenic and non-cyanogenic (the word cyanogenic derives from the bluish color of affected patients' skin). The severity of the damage varies from case to case.

Non-cyanogenic congenital heart disease can result from various malformations: Holes in the heart or septal defects (atrial septal defect (ASD), ventricular septal defect (VSD)) and obstruction of blood flow (stenosis, atresia, deficiency). While in some cases, medication may be sufficient to treat a patient, in many cases, defective heart valves need to be repaired. As the availability of human heart valves is extremely limited (sourced from a cadaver), or even non-existent in children for size reasons, repairs generally involve the use of valves made from biological materials, notably animal pericardium (particularly bovine) and usually chemically treated (e.g. glutaraldehyde fixation) to avoid rejection and rapid degradation. This strategy underpins the design of all the so-called biological valves currently on the market, since they are of animal origin and mounted on a synthetic support structure, often including a type of stent [20]. However, this fixation causes a chronic, non-specific inflammatory reaction known as “foreign body reaction” (FBR), similar to that observed when synthetic biomaterials are implanted, and the main cause of implant complications. However, FBR is much less intense than specific rejection directed at intact animal tissues, hence the benefit of fixation. Moreover, by denaturing the proteins, binding renders them insensitive to host enzymes, which are normally capable of rapidly degrading them. The use of human tissue obtained from the patient's pericardium, and therefore not requiring chemical fixation, is a possibility that has been explored [21]. This seems very promising, but availability, quality variability and quality itself, fragility, additional operating time, and the consequences of transplantation for the donor site are all variables that can complicate this approach. All these complications would be eliminated by using a tissue-engineered biological membrane such as the one proposed in this invention.

The use of a “bioengineered film”, assumed to be obtained from a biodegradable polymeric material or from proteins purified and then reconstituted as a matrix, has also been evoked in the case of [20]. First of all, the use of the term “film” suggests to the person skilled in the art a very thin covering (Le Petit Robert dictionary: “Very thin layer (of a material)”). A literature review in PubMed confirms that the use of this term generally refers to very small layers of material. Such films do not appear to be suitable, as they are far too thin compared to a sheet of tissue to provide sufficient puncture resistance. What's more, this type of material is used in an approach based on the delicate balance between the phenomena of material degradation and the creation of a new tissue. This balance is very difficult to control, given that these phenomena vary greatly from one individual to another. But more important is the fact that the person skilled in the art will understand “bioengineered” to mean: A structure made of either chemically treated animal (or even cadaver) tissue or a structure made of proteins solubilized/purified from animal/human tissue extracellular matrix which are then reassembled by various physical/chemical processes. This perception is confirmed on reading Patent Application US20030229394A1 [22] where the authors divide matrices made from extracellular matrix proteins (mainly collagen) into 1) a natural tissue or 2) a “synthetic” collagen-based matrix. Here, “synthetic” does not mean “made from polymer”, as is often the case, but “made from proteins extracted from natural tissues”, as described in paragraph 79, where “synthetic tissue matrices” are described.

Tetralogy of Fallot is one of the most common forms of cyanogenic congenital heart disease. It accounts for 7 to 10% of newborns with congenital heart disease [1, 2], and combines pulmonary valve stenosis, right ventricular hypertrophy, ventricular septal defect (VSD), and overriding aorta. These abnormalities will affect the structure and capacity of the pulsatile heart, leading to stenosis of the right ventricular outflow tract (RVOT), and ultimately to oxygen desaturation in arterial blood. Surgical repair involves closing the VSD and removing the RVOT stenosis at around 6 months of age. In over 70% of cases, RVOT stenosis is corrected by placement of a transannular patch, which unfortunately leads to pulmonary valve leakage [3, 4]. Left untreated, this pulmonary valve leakage leads to right ventricular dilatation and dysfunction, which will be associated with ventricular arrhythmias and an increased risk of sudden death in adulthood. The use of stent-mounted biological valves or industry-supplied mechanical valves in this infant population remains totally impossible [5]. Techniques have been developed to limit pulmonary valve leakage, such as the use of a monocuspid valve [6]. This valve can be made from biological materials, notably by using chemically treated bovine pericardium to prevent rejection [5] or synthetic polytetrafluoroethylene (PTFE) membranes [6]. However, these materials are subject to limitations. Although relatively flexible, extremely strong and easy to handle, glutaraldehyde-treated bovine pericardium and synthetic membranes are associated with thromboembolic complications and/or infective endocarditis [7-9]. What's more, these materials are recognized as foreign bodies that cause chronic inflammatory reactions. Polytetrafluoroethylene (PTFE) membranes, like all synthetic materials, are also a cause of serious infections. Finally, current options have shown ineffective short—and medium-term results in limiting pulmonary valve leakage.

There is therefore a real clinical need to develop a new material for repairing valves such as heart valves, without the adverse effects described.

The inventors propose to use a completely biological biomaterial composed of an extracellular matrix (ECM) secreted by human dermal fibroblasts, which is truly biocompatible [10-13]. Its remarkable biocompatibility stems from the structure of the ECM, which represents a different advantage in addition to that of the biological or human nature of the proteins of which it is composed. During implantation, the body's immune system can recognize the foreign origin of a protein, thanks to its “adaptive” immune system, but it can also recognize an abnormal (or “denatured”) ECM structure, thanks to its “innate” immune system. As a result, when the ECM is damaged by physical, thermal or infectious trauma, the body can rapidly digest the damaged ECM, using macrophage-type cells, to enable specialized fibroblast-type cells to rebuild an ECM. This is the mechanism that comes into play when a collagen matrix produced by physical/chemical methods is implanted into an organism. It will be rapidly degraded by the innate immune system, as it does not have a “physiological” structure but rather a denatured one [23-25]. The fact that the ECM is produced by cultured cells in a laboratory, and not chemically treated afterwards, means that the structure of the ECM is close enough to the physiological structure to enable it to be “accepted” by the host, or, in other words, not to trigger an innate immune response that would destroy it. It is this structural feature that differentiates it from all other matrices produced by physical/chemical methods from proteins extracted from tissue (human or animal). Moreover, this ECM, produced in sheet form on the surface of the culture flask, is easy to handle and mechanically robust after 6 to 8 weeks of culture [13,14]. This robustness, achieved without chemical treatment (such as cross-linking, for example), is also attributable to the ECM being formed by cultured cells, which produce a matrix of densely organized collagen fibrils combined with numerous other matrix proteins [26-27]. Finally, it should be noted that the structure of this ECM produced by cells in the laboratory is also different from that of the ECM obtained from tissue taken directly from a mammal. This is because tissues harvested in situ contain numerous structures (hairs, nerves, blood and lymphatic vessels, glands, membranes, limiters, etc.) that are absent from the ECM sheets produced in vitro. What's more, these harvested tissues contain numerous cell types, whereas the ECM sheets produced in vitro contain only a single cell type (typically fibroblasts). As a result, the leaflet produced in vitro is much more homogeneous, in terms of structure and composition, than tissue harvested in vivo. This can have both mechanical and biological benefits. What's more, because of its in vitro preparation mode, the leaflet offers greater reproducibility, which can be advantageous from the point of view of quality control in a commercial production context. As a result, the structure of this material is very special, distinguishing it from other materials used to produce valve leaflets.

This biomaterial has already been used in vascular applications in humans [15-18]. In this context, sheets of ECM were rolled up and fused to form vessels, which were then implanted in patients suffering from end-stage renal failure [17, 18]. Implantation results have shown a high rate of transluminal patency (up to 3 years) [17, 18]. Furthermore, this work demonstrated that the ECM sheet constructed without synthetic components can actually integrate with native tissue, be remodeled by host cells and be resistant to infection. Its biological structure, human origin and absence of chemical treatments (e.g. glutaraldehyde fixation) have enabled this biomaterial to be accepted by patients without risk of rejection, rapid degradation or chronic inflammation, unlike ECMs derived from animal tissues, which are always chemically treated to mask their xenogenic origin and slow down the degradative action of host enzymes secreted to destroy denatured matrices. Furthermore, data have shown that biomaterial produced by allogeneic fibroblasts (from a donor different from the recipient) does not induce a specific adaptive immune response [15]. In addition, a new generation of biological threads has been produced, by cutting a sheet of ECM into a plurality of ribbons. These ribbons can be twisted to form denser threads with different mechanical properties than ribbons [14]. Recent work has demonstrated the feasibility of using these ECM threads or ribbons as sutures for skin wound closure [14],

This biomaterial with a puncture resistance of up to 2-6 kgf [26] has also been used in the construction of a valve implant comprising a tubular structure and at least two pieces of ECM sheet (cusps or leaflets) connected to said structure; the sheet being as defined above (see “single-layer tissue sheet” from International Application WO 20123/142879) [19]. This biological implant has never been proposed for leaflet repair in children with congenital heart disease, particularly tetralogy of Fallot.

### DESCRIPTION OF THE INVENTION

The present invention precisely addresses this need by proposing a biological implant comprising or consisting of the combination of a sheet of tissue made up of an extracellular matrix (ECM) secreted by cells, preferably human cells, in culture and possibly the cells themselves (hereinafter also referred to as an ECM sheet), with modified biological threads or ribbons that are synthetic and/or derived from the same biological material. This biological implant is used to create a pocket-type leaflet implant into which a biological sample and/or active ingredient can be inserted prior to implantation, in order to optimize tissue regeneration.

To this end, the Inventors have developed a method for manufacturing a completely biological leaflet implant (FIG. 1) from an ECM sheet as defined above, from which they have cut a piece that has been folded once on itself to form a pocket-type leaflet. In parallel, ECM sheets were also cut into ribbons to produce biological ECM threads. Once the piece and the ECM threads or ribbons had been obtained, optionally, native pulmonary valve cusps or leaflets containing living cells were extracted, particularized and positioned in the pocket-shaped leaflet. The pocket was then closed and the construction sutured at the pulmonary outflow tract using biological or synthetic sutures or tapes. The space between the two sheet layers formed by the pocket closure has not been sealed/fused to allow it to be filled with cells, tissue fragments or active ingredients. This innovative combination has enabled the production of a fully biological leaflet implant that can be readily accepted by the host, can be colonized by host cells, can be remodeled after implantation (e.g. by the growing child treated for tetralogy of Fallot), and can change with the patient. In other words, this leaflet implant has the potential to grow, which differs from prior art implants (e.g. with stent, fixed animal pericardium, polymer film or bioengineered film made from isolated and reconstituted proteins) that require removal and replacement via open-heart surgery associated with a high mortality rate. The leaflet implant according to the invention therefore has potential for pediatric applications.

The human nature and quasi-native structure of this ECM obviates the need for fixation to prevent its degradation or to reinforce it, enabling the leaflet implant according to the invention to be accepted by the recipient patient, without rejection or chronic inflammation with visible vascularization at two weeks; this promises superior efficacy and durability to valves or leaflets made from materials of animal or synthetic origin. Using reconstituted ECM would lead to complete degradation within a few weeks and device failure, or would require chemical treatments that would make it a source of chronic inflammation, which is a source of complication and failure of leaflet devices.

### PRODUCTION OF A SHEET OF EXTRACELLULAR MATRIX (ECM) SECRETED BY CELLS

Cells are seeded on a substrate with culture medium to generate an ECM sheet after a long culture period. The ECM sheet is optionally treated for storage. The ECM sheet may or may not be detached from the substrate using an anchoring device. This device prevents tissue contraction during cultivation and treatment for storage. Finally, the ECM sheet is cut into a piece of ECM sheet.

The cells can be of human origin. The cells can be autologous or allogeneic. The cell type may be, for example, mesenchymal stem cells, induced pluripotent stem cells, primary adult mesenchymal cells (such as skin fibroblasts), immortalized cells, or a mixture thereof.

The seeding concentration depends on the cell type and can range from 1000 cells/cm² to 100,000 cells/cm².

The substrate can be selected from: a standard plastic culture flask, a heat-sensitive culture flask, a different or similar ECM (e.g. autologous human, allogeneic human, xenogeneic ECM, etc.), a treated biological membrane (e.g. chemically treated pericardium of various origins, amniotic membrane, etc.), a synthetic membrane (e.g. polytetrafluoroethylene, dacron, silicone, etc.), a hydrogel (e.g. collagen, alginate, fibrin, etc.), a hybrid of the above substrates. The substrate can be coated with adhesion proteins (e.g. RGD peptides, fibrin, etc.) or gelatin to facilitate cell adhesion. The substrate can have different shapes (e.g. customized, round, rectangular, square, triangular, octagonal, pentagonal, etc.) to obtain the desired ECM sheet format.

The culture medium contains, but is not limited to, ascorbic compounds and serum for ECM production and assembly. Its composition depends on cell type and is changed typically three times a week. The cultivation period varies from 4 to 24 weeks, preferably from 16 to 20 weeks, and more preferably from 6 to 8 weeks.

For storage, the ECM can be untreated, dehydrated, devitalized (a method consisting in freezing the ECM sheet at −80° C., thawing it at room temperature, dehydrating it preferably overnight at room temperature under the sterile flow of a fume hood and finally rehydrating it at room temperature before use), decellularized, treated with a glutaraldehyde solution, or a combination thereof. Decellularized ECM can be recellularized with the cells described above, or endothelialized using autologous or allogeneic cells (e.g. umbilical cord vein endothelial cells (HUVECs), adipose tissue-derived stromal vascular fraction (SVF) cells, microvascular cells, etc.). Depending on the treatment, the ECM can be stored at −80° C.,−20° C., 4° C. or at room temperature. The anchoring device can be a sterile stainless steel holder, a sterile plastic holder, a sterile paper holder, and depends on the culture flask.

This ECM sheet is neither a natural tissue nor a synthetic tissue produced from proteins extracted from natural tissue and reconstituted into a tissue derived from tissue bioengineering. This ECM sheet is assembled by cells in culture. The result is an unfixed sheet of human ECM with a physiological structure. For these reasons, the ECM sheet possesses significant mechanical strength, does not cause immune/inflammatory reactions, and can interact normally with recipient cells to enable a slow remodeling rather than a process of degradation.

### PRODUCING THREADS OR RIBBONS FROM ECM

The ECM sheet is cut into ribbons that can be twisted (e.g. from 0 to 10 turns/cm) to form wires. The ECM sheet can be cut using a machine with spaced circular blades, a laser system, an ultrasound system, an electric arc system, a scalpel blade, or scissors. The ribbons can be from 0.1 to 10 mm wide, with the length depending on the substrate used. The ribbons can be formed directly onto culture flasks in the desired format. The ribbons can be twisted to modify their properties. At least two ribbons can be joined together using a twisting method, biological glue, cultured ECM deposition, or a combination thereof, to improve their mechanical properties. The threads or ribbons can then be mounted on a surgical needle (e.g. knotted at the eye; the eye can be crimped to secure the thread or ribbon to the needle).

For storage, threads or ribbons can be untreated, dehydrated, devitalized, decellularized, glutaraldehyde-treated, or a combination thereof. Decellularized threads or ribbons can be recellularized with the cells described above, or endothelialized using autologous or allogeneic cells (e.g. HUVECs, cells from the vascular stromal fraction of blood, microvascular cells, etc.). Depending on the treatment, the threads or ribbons can be stored at −80° C., −20° C., 4° C. or at room temperature.

These threads or ribbons are unlikely to cause inflammatory reactions, and can be reshaped. This can help avoid complications and allow them to grow with the child, unlike traditional sutures generally made of permanent plastics (e.g. polypropylene).

### PARTICLE PRODUCTION AND INSERTION

Particles containing living cells can be particularized and positioned inside the pocket-shaped leaflet implant.

For example, particles can be derived from: Native pulmonary valve leaflet, native dermis (autologous, allogeneic or xenogeneic), tissue-engineered ECM, any tissue-engineered matrix, cell suspension (primary cells, mesenchymal stem cells, induced pluripotent stem cells (iPSCs), etc.), cell aggregate (e.g. spheroids, organoids), extracellular vesicle or active ingredient (e.g. drug), or a combination thereof.

The particles can be obtained using a machine with spaced circular blades, a laser system, an ultrasound system, an electric arc system, a scalpel blade, scissors, or a grinder.

The particles can be placed inside the bag using a spoon (surgical equipment), pipette, or syringe.

Thus, according to one aspect, the invention relates to a pocket-type leaflet implant comprising:
a piece of a tissue sheet consisting of an extracellular matrix secreted by cells, preferably human, in culture and optionally cells themselves, folded on itself once so as to form a pocket area, wherein all or some of the opposite edges of said folded piece are joined together.

For the purposes of this invention, “leaflet” means that part of a valve which consists of a sheet of tissue that moves with a fluid (preferably blood) to allow it to flow in one direction and prevents it from flowing in the other. There may be one, but more usually two or three, or even more leaflets in a valve.

For the purposes of the present invention, a “pocket-type leaflet” is a leaflet comprising a hollow structure for retaining one or more objects that can be placed therein, e.g. cells, small pieces of tissue, biomaterial particles, etc.

For the purposes of this invention, “tissue sheet consisting of an extracellular matrix secreted by cells, preferably human, in culture” means the deposit of insoluble proteins which accumulates on the inner surface of the bottom wall of a container in which cells are cultured under conditions which favor this deposit. More specifically, it is a tissue sheet when this deposit is detached from the surface.

For the purposes of this invention, “pocket area” means the empty space (hollow structure) created by folding a piece of ECM sheet and closing all or part of the opposite edges of said piece.

For the purposes of this invention, “opposite edges” are defined as the edges of a piece of ECM sheet whose adjacent flat portions are essentially parallel and rest against each other in such a way that they can be joined.

According to a particular embodiment of the present invention, all or part of the opposite edges of said folded piece are joined together by suture with biologically modified (silk, chitosan, collagen, animal intestinal wall, etc.), synthetic and/or tissue-sheet-derived thread or ribbon as defined herein.

For the purposes of this invention, “(synthetic) thread or ribbon” means a thread or ribbon made from a chemically synthesized material used for surgery or the preparation of medical devices.

According to a particular embodiment of the present invention, said implant comprises within the pocket area a biological sample and/or an active ingredient.

A “biological sample” in the sense of the present invention is preferably a portion of the patient's dysfunctional leaflet removed and cut into small pieces in the operating room using a common surgical tool (e.g. scissors). The size of the pieces may vary. These pieces are the biological samples that will be placed in the valve pocket area to provide a cell population to recolonize the tissue. Other tissues can be used, such as the dermis, connective tissue, bone marrow, blood vessels, blood, adipose tissue, etc. Other cell sources are envisaged, such as cells from the same patient, but which have been cultured in vitro and may have undergone different treatments, such as differentiation or dedifferentiation. These cells can come from different parts of the body. They can be assembled into larger or smaller clusters (organoids). Several cell types can be combined. Cells can also be combined with one or more biomaterials (biological or synthetic) to enhance their survival or function, according to a wide range of delivery strategies. Cells can also come from another individual (allogeneic) or species (xenogeneic). The above options can be combined.

For the purposes of the present invention, “active ingredient” means a pharmacological (non-living) agent which will have a positive effect on leaflet function by, for example, promoting the migration or proliferation of the patient's own cells to achieve faster or more effective tissue recolonization. The pharmacological agent may also have an anti-thrombotic effect.

According to a particular embodiment of the present invention, the biological sample comes from at least one leaflet of a valve of a subject.

A “valve” in the sense of the present invention is a tubular structure which has, in its lumen, one or more leaflets which allow a fluid to flow in one direction but not the other.

According to another aspect, the present invention relates to a method for manufacturing a leaflet implant according to the present invention, said method comprising:
a) cultivation of a sheet of tissue as defined above;
b) cutting a piece of said sheet of tissue obtained in step a);
c) folding said piece from step b) onto itself once to form a pocket area;
d) joining all or some of the opposite edges of said folded piece from step c).

According to a particular embodiment of the method of the present invention, the joining of step d) is achieved by suturing with biologically modified, synthetic and/or tissue-sheet-derived thread or ribbon as defined in the present invention.

According to a particular embodiment of the method of the present invention, said method further comprises a step d′) of filling the pocket area with a biological sample and/or an active ingredient, prior to the complete joining of step d).

According to a particular embodiment of the method of present invention, the biological tissue sample comes from at least one leaflet of a valve of a subject.

According to yet another aspect of the present invention, the leaflet implant according to the present invention is for use as a medicament.

According to yet another aspect of the present invention, the leaflet implant according to the present invention is for use in the treatment of a pathology selected from congenital heart disease, cardiac, venous and lymphatic valvulopathies.

According to a particular embodiment of the present invention, the congenital heart disease is tetralogy of Fallot.

Other advantages to those disclosed in the present Application may also be apparent to those skilled in the art upon reading the examples below, given by way of illustration.

### BRIEF DESCRIPTION OF THE FIGURES

FIG. 1 shows the method for manufacturing a valve leaflet implant according to the invention.

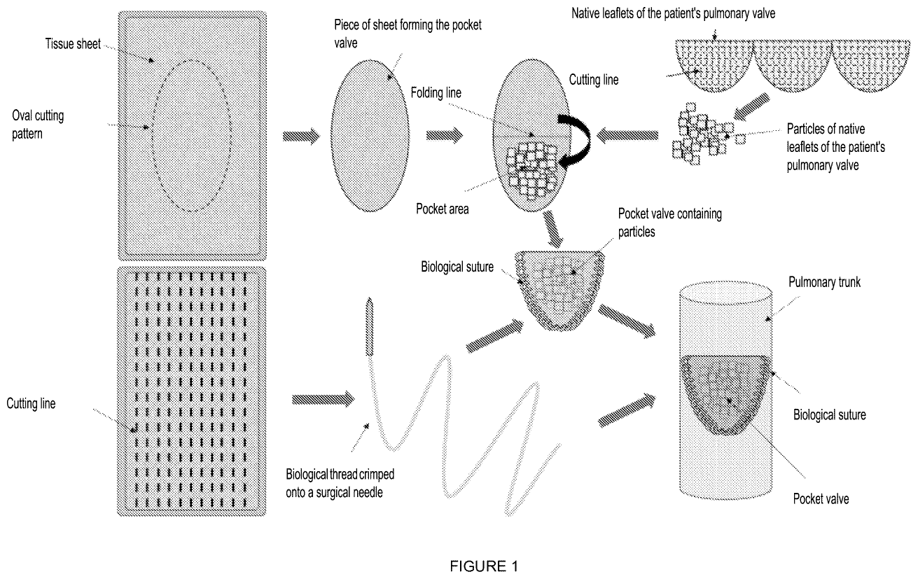

FIG. 2 shows the pattern used to cut the 2.4 m-long, 5 mm-wide biological ribbon.

FIG. 3 shows the steps involved in crimping the biological suture thread. The hydrated biological ribbon is twisted at 5 revolutions/cm over a length of about 2 cm at one end to form a thread. During this stage, the twisted area is gently massaged until it is completely dry, to distribute the twists and obtain a dry thread with an even diameter. This homogeneous zone is then inserted into the eye of a surgical needle to be crimped.

FIG. 4 shows a ready-to-use hydrated biological suture.

FIGS. 5A-5E show the steps required to produce the pocket-shaped leaflet. (A) The sheet of tissue made of ECM secreted by cultured cells (hydrated) is placed on a cutting surface including a pattern (black solid line) of oval shape (length: 8 cm and width: 3 cm) to be dried. (B) Once dry, the sheet of tissue is cut to the pattern. (C) The tissue is rehydrated with a sterile water solution. (D-E) Once rehydrated, the piece of sheet of tissue can be detached from the cutting support and folded onto itself along the dotted black line to form the pocket-shaped leaflet.

FIG. 6 shows the pocket-shaped leaflet closed with an overlock stitch using the biological suture shown in FIG. 4.

### EXAMPLES

### Example 1: Method for Manufacturing a Completely Biological Leaflet Implant and Features Thereof

This example describes an approach to manufacturing a completely biological leaflet implant. In this example, the resulting leaflet is composed of non-living (devitalized) tissue and produced sterile, which precludes the use of a terminal sterilization or fixation step. All assembly steps are carried out in a sterile environment, using sterile fluids and instruments. This description is not intended to limit the scope of this invention with respect to the method of producing the leaflet, cell types, cell source, cell age, cell line, culture conditions, shape of the sheet or piece of sheet, number of sheets or pieces of sheets, suture material, suture method or intended use of the leaflet. The skilled person will readily understand that various modifications can be made to the method without departing from the scope and spirit of the invention.

In this example, tissue sheets were obtained by culturing normal human skin fibroblasts in T-225 cm² flasks. For each flask, cells were seeded at a density of 10,000 cells/cm² and grown in a culture medium consisting of DMEM-F12, bovine serum (20%) and sodium ascorbate (500 mM). The whole set was placed in an incubator with a 37° C. atmosphere composed of 5% CO2 and 95% air. The culture medium was changed three times a week. After around three days, L-shaped rods made of 304 stainless steel were produced. After a 16-week culture period, the culture flask was cut in half lengthwise with a hot wire, and the sheet was taken out of the flask using the inner frame (an anchoring system which also serves to handle the sheet) to be used in the manufacture of the biological thread and pocket-shaped leaflet. A mechanical study established that this sheet was capable of withstanding a perforation force of around 2300 grams-force.

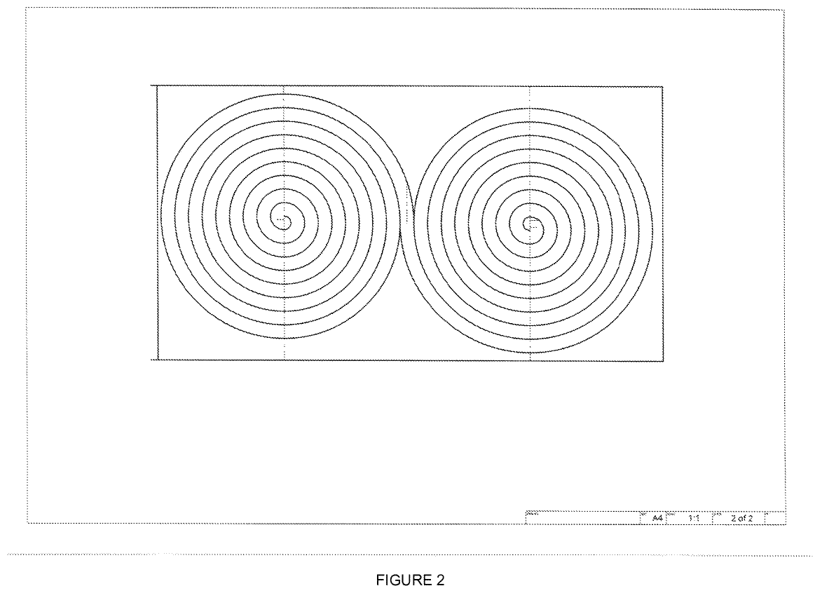

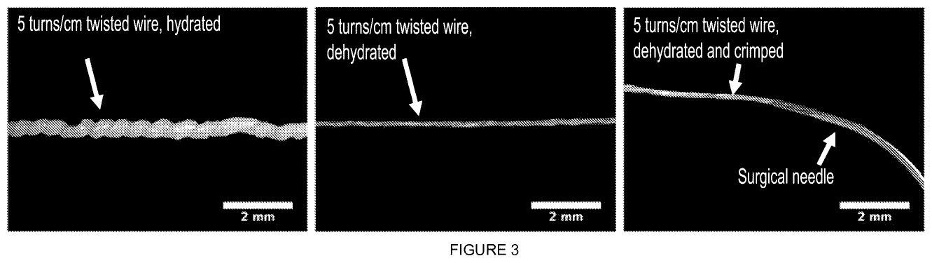

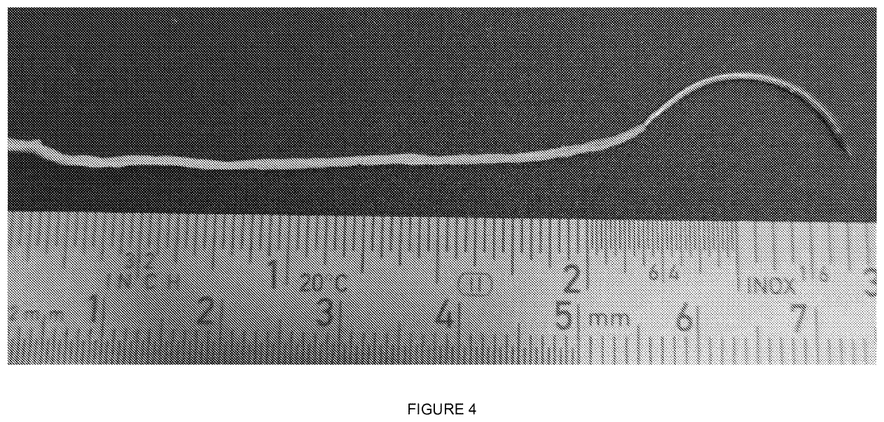

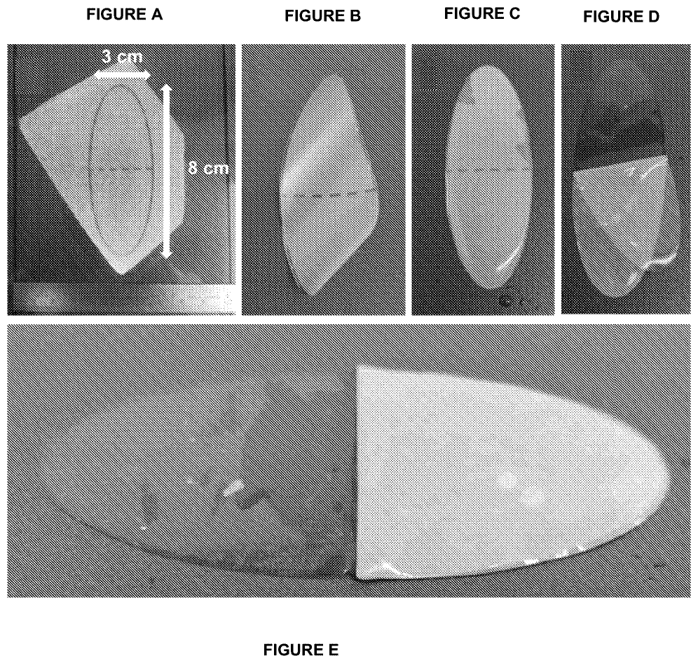

To produce the biological suture, a sheet of moist tissue was placed on a cutting surface including a double spiral pattern to produce a ribbon 5 mm wide and 2.4 m long (FIG. 2). The sheet of tissue was then dried on the cutting surface for at least 2 hours in a laminar flow hood. Once dried, the ribbon was then cut manually using surgical curved scissors and then rehydrated to separate it from the cutting surface. One end of the ribbon was then twisted on itself with a rotary motor at a rate of 5 revolutions per cm over a length of 2 cm to form a thread (FIG. 3). This twisted area was gently massaged during twisting and until the end was completely dry, reducing the thread diameter as much as possible (FIG. 4). This dry, thin area was then inserted into the eye of a surgical needle and the mounting was crimped using a pneumatic crimper (FIG. 4). The crimped bio-thread was then wrapped dry around a silicone tube and placed in an Eppendorf tube to be preserved at −80° C. until use. For use, the biological suture was rehydrated with water for at least 10 minutes (FIG. 5).

To obtain particles containing living cells, the patient's native pulmonary valve leaflets were surgically resected and then separated using a scalpel blade. The particles were then drawn into a syringe for delivery into the pocket-shaped leaflet.

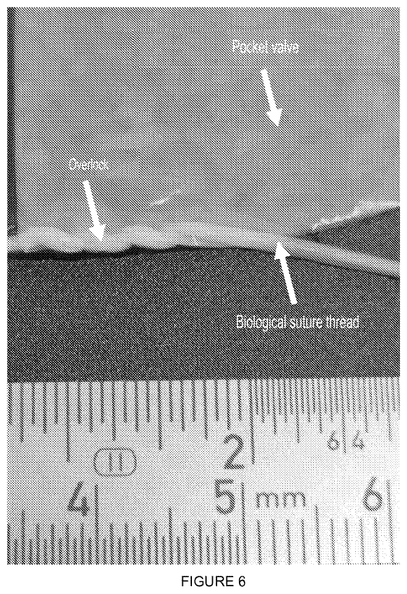

To form the pocket-shaped leaflet, a sheet of moist tissue was placed on a cutting surface including an oval-shaped pattern (length: 8 cm, width: 3 cm) and dried in a laminar flow hood for at least 2 hours. The dried sheet was then cut to the pattern using curved scissors. Once cut, the tissue was rehydrated with water for at least 30 minutes and the particles containing living cells were placed on half the surface of the piece of sheet forming the pocket-shaped leaflet using the syringe. Finally, the piece of sheet was folded on itself along the short axis (3 cm) of the oval shape to give a pocket-type leaflet with the following dimensions: height 4 cm and width 3 cm. Biological suture was then used to close the free edges of the pocket-type leaflet using an “overlock” suturing method (FIG. 6). In order to prevent patient pulmonary leak created as a result of the widening of the pulmonary outflow tract during surgical correction of Tetralogy of Fallot, the leaflet would ultimately be sutured approximately 1 cm above the pulmonary annulus using a biological suture or tape similar to that used to close the pocket, or a synthetic suture or tape.

### LIST OF REFERENCES

1. Apitz et al, Lancet, 374: 1462-1471, 2009
2. Warnes et al, ACC Cardiosource Rev. J., 17: 19-23, 2008
3. Singh et al, Congenit. Heart Dis, 13: 935-943, 2018
4. Scavo et al, ASAIO J., 44: M480-M485, 1998
5. Gundry et al, Pediatr. Card. Surg. Annu. Semin. Thorac. Cardiovasc. Surg, 2: 77-82, 1999
6. Turrentine et al, Ann. Thorac. Surg, 73: 871-880, 2002
7. Mather et al, Cardiovasc. Pathol. 21: e35-e38, 2012
8. Shrivastava et al, Int. J. Cardiol., 25: 27-31, 1989
9. Bhukar et al, J. Card. Surg, 32: 476-478, 2017
10. L'Heureux et al, FASEB J., 12: 47-56, 1998
11. Peck et al, Cells Tissues Organs, 195: 144-158, 2011
12. Peck et al, Mater. Today., 14: 218-224, 2011
13. Magnan et al, Acta Biomater, 82: 56-67, 2018
14. Magnan et al, Acta Biomater, 105: 111-120, 2020
15. Wystrychowski et al, J. Vase. Surg, 60: 1353-1357, 2014
16. Wystrychowski et al, J. Vase. Access, 12: 67-70, 2011
17. L'Heureux et al, N; Engl. J. Med., 357: 1451-1453, 2007
18. McAllister et al., Lancet, 373: 1440-1446, 2009
19. International application WO 2013/142879
20. International application WO 2007/123658
21. Pande et al, Ann Card Anaesth, 25: 164-170, 2022, doi: 10.4103/aca.aca_22_21
22. Patent application US20030229394
23. Magnan et al, Biomaterials, 273: 120815, 2021, doi: 10.1016/j.biomaterials.2021.120815
24. Wang et al, J Bioact Compatible Polym, 21: 39-54, 2006, doi: 10.1177/0883911506060201
25. Srivastava et al, Biomaterials, 11: 155-161, 1990
26. Magnan et al, Acta Biomater, 82: 56-67, 2018, doi: 10.1016/j.actbio.2018.10.006
27. Kawecki et al, Biomaterials Science, 2022, doi: 10.1039/d1 bm01933c

1. Pocket-type valve leaflet implant comprising:
a piece of a tissue sheet consisting of an extracellular matrix secreted by cells, preferably human, in culture and optionally cells themselves, folded on itself once so as to form a pocket area, wherein all or some of the opposite edges of said folded piece are joined together, with the understanding that said cells are not human embryonic stem cells, genetically modified or not.

2. The leaflet implant according to claim 1, wherein all or some of the opposite edges of said folded piece are joined together by suture with biologically modified, synthetic and/or tissue-sheet-derived thread or ribbon.

3. The leaflet implant according to claim 1, said implant comprising inside the pocket area a biological sample and/or an active ingredient, with the understanding that the biological sample is not human embryonic stem cells, genetically modified or not.

4. The leaflet implant according to claim 3, wherein the biological sample comes from at least one leaflet of a valve of a subject.

5. A method for manufacturing a leaflet implant according to claim 1, said method comprising:
a) cultivating the sheet of tissue;
b) cutting a piece of said sheet of tissue obtained in step a);
c) folding said piece from step b) onto itself once to form a pocket area;
d) joining all or some of the opposite edges of said folded piece from step c).

6. The method according claim 5, where the joining in step d) is made by suturing with modified, synthetic and/or tissue-sheet-derived biological thread or ribbon.

7. The method according to claim 5, said method further comprising a step d′) of filling the pocket area with a previously collected biological sample and/or active ingredient, prior to the complete completion of step d).

8. The method according to claim 7, wherein the biological tissue sample previously taken comes from at least one valve leaflet of a subject.
